(19) United States
(12) Patent Application Publication
(10) Pub. No.: US 2023/0165552 A1
(43) Pub. Date: Jun. 1, 2023

(54) MEDICAL IMAGE PROCESSING APPARATUS, X-RAY DIAGNOSTIC APPARATUS, AND STORAGE MEDIUM

(71) Applicant: CANON MEDICAL SYSTEMS CORPORATION, Tochigi (JP)

(72) Inventors: Hisato TAKEMOTO, Nasushiobara (JP); Kazuhiro TANIYAMA, Otawara (JP); Tomoki FUJITO, Nasushiobara (JP)

(73) Assignee: CANON MEDICAL SYSTEMS CORPORATION, Tochigi (JP)

(21) Appl. No.: 18/058,964

(22) Filed: Nov. 28, 2022

(30) Foreign Application Priority Data
Nov. 30, 2021 (JP) 2021-194426

(51) Int. Cl. A61B 6/00; A61B 6/12

(52) U.S. Cl. CPC A61B 6/4441; A61B 6/12; G06T 2207/10116

(57) ABSTRACT

A medical image processing apparatus according to an embodiment includes processing circuitry. The processing circuitry obtains X-ray images that are sequentially collected during a partial period of time in a cardiac phase of a subject who has a device inserted into the body. Then, the processing circuitry identifies a characteristic region of the device captured in a plurality of obtained X-ray images, and performs registration in which the position of the characteristic region identified in a reference image, which is one of the plurality of X-ray images, serves as the reference position, and in which the position of the characteristic region identified in the plurality of X-ray images collected after the reference image is adjusted based on the reference position.

### CROSS-REFERENCE TO RELATED APPLICATIONS

This application is based upon and claims the benefit of priority from Japanese Patent Application No. 2021-194426, filed on Nov. 30, 2021; the entire contents of which are incorporated herein by reference.

### FIELD

Embodiments described herein relate generally to a medical image processing apparatus, an X-ray diagnostic apparatus, and a storage medium.

### BACKGROUND

In the vascular intervention treatment, a device such as a catheter, a guide wire, or a stent is inserted into a blood vessel and is accurately moved to the target region for treatment. Then, a procedure is performed for expanding the device so as to mechanically expand the strictured area, or a procedure is performed for placing the device inside the body. In this treatment approach, the position for performing a procedure needs to be decided, and the completion of the procedure needs to be confirmed. Usually, the doctor refers to X-ray images that are generated and displayed in real time in an X-ray diagnostic apparatus, and performs the procedure as well as confirms the completion of the procedure. For example, at two positions (sometimes one position) on a guide wire, a radio-opaque metal is attached as markers for indicating the position of the balloon or the position of the stent. Thus, the doctor refers to the markers that are visualized in the X-ray images displayed in a monitor, and decides on the position for performing a procedure. Moreover, the doctor refers to the device visualized in the X-ray images and confirms the completion of the procedure.

However, in the vascular intervention treatment of a pulsatory organ such as the heart, there are times when the position of the device changes within the field of view due to cardiac pulsation, or due to body motion, or due to breathing. For that reason, if a plurality of X-ray images is collected after the insertion of a device, then the position at which the device is visualized changes in each X-ray image, thereby resulting in a decline in the visibility of the device. In that regard, a technology is known in which, regarding the X-ray images that are sequentially collected and displayed in a monitor, the two markers that are visualized in each X-ray image are detected; deformation and registration is performed with respect to the X-ray images with the aim of ensuring that the positions of the two markers in each X-ray image are same as the positions in the past images; and a video display is so performed that the device virtually appears to be still. Moreover, a technology is known in which, for example, the arithmetic mean is taken of a plurality of X-ray images corrected to have the same positions of the two markers, so that the device gets displayed in a highlighted manner.

### BRIEF DESCRIPTION OF THE DRAWINGS

FIG. 1 is a block diagram illustrating an exemplary configuration of a medical image processing apparatus according to a first embodiment;

FIG. 2 is a diagram illustrating an example of a registration operation and an addition operation performed with respect to the X-ray images in which a stent is visualized;

FIG. 3 is a sequence diagram illustrating the sequence of operations performed according to the first embodiment;

FIG. 4 is a diagram illustrating an example of the correspondence relationship between the electrocardiogram and the periodic movement of the heart according to the first embodiment;

FIG. 5 is a diagram illustrating an example of the correspondence relationship between the irradiation period and the pulse irradiation according to the first embodiment;

FIG. 6 is a diagram illustrating an example of the registration operation and the addition operation performed according to the first embodiment;

FIG. 7 is a diagram illustrating an example of the details of the registration operation performed according to the first embodiment;

FIG. 8 is diagram illustrating an example of the details of the registration operation performed according to the first embodiment; and

FIG. 9 is a block diagram illustrating an exemplary configuration of the X-ray diagnostic apparatus according to another embodiment.

### DETAILED DESCRIPTION

A medical image processing apparatus according to an embodiment includes processing circuitry. The processing circuitry obtains X-ray images that are sequentially collected during a partial period of time in a cardiac phase of a subject who has a device inserted into the body. Then, the processing circuitry identifies a characteristic region of the device captured in a plurality of obtained X-ray images, and performs registration in which the position of the characteristic region identified in a reference image, which is one of the plurality of X-ray images, serves as the reference position, and in which the position of the characteristic region identified in the plurality of X-ray images collected after the reference image is adjusted based on the reference position. Exemplary embodiments of a medical image processing apparatus, an X-ray diagnostic apparatus, and a storage medium are described below in detail with reference to the accompanying drawings. However, the medical image processing apparatus, the X-ray diagnostic apparatus, and the storage medium according to the application concerned are not limited by the embodiments described below.

First Embodiment

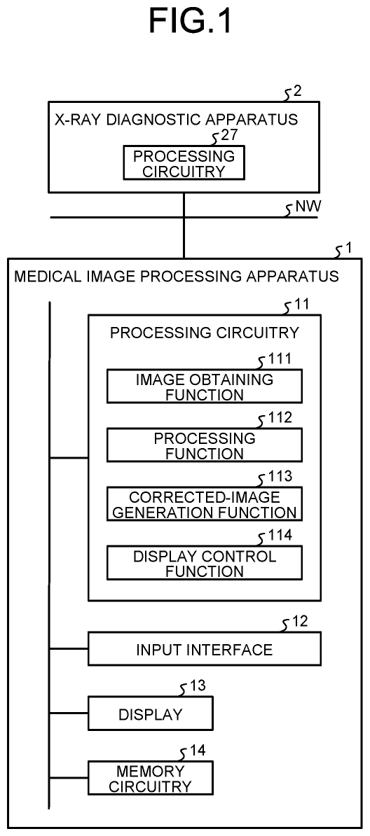

Given below is the explanation of a configuration of a medical image processing apparatus according to a first embodiment. FIG. 1 is a block diagram illustrating an exemplary configuration of a medical image processing apparatus 1 according to the first embodiment. As illustrated in FIG. 1, the medical image processing apparatus 1 includes processing circuitry 11, an input interface 12, a display 13, and memory circuitry 14. The medical image processing apparatus 1 is connected to an X-ray diagnostic apparatus 2 via a network.

The X-ray diagnostic apparatus 2 includes processing circuitry 27, and performs X-ray irradiation under the control of the processing circuitry 27. Then, the X-ray diagnostic apparatus 2 collects X-ray image data. For example, the X-ray diagnostic apparatus 2 according to the first embodiment collects X-ray image data based on electrocardiogram information. Meanwhile, the details of the X-ray diagnostic apparatus 2 are explained later.

The processing circuitry 11 reads a computer program stored in the memory circuitry 14 and executes the computer program. As a result, the processing circuitry 11 functions as an image obtaining function 111, a processing function 112, a corrected-image generation function 113, and a display control function 114. The processing circuitry 11 is configured using, for example, a processor. The processing circuitry 11 obtains X-ray image data from the X-ray diagnostic apparatus 2 and performs image processing. The image obtaining function 111 represents an example of an image obtaining unit. The processing circuitry 11 represents an example of processing circuitry.

The image obtaining function 111 obtains X-ray image data via a network. The processing function 112 performs image processing with respect to the X-ray image data and generates X-ray images. Then, the processing function 112 stores the generated X-ray images in the memory circuitry 14. The corrected-image generation function 113 generates a corrected image based on an X-ray image. The display control function 114 displays various X-ray images in the display 13. Regarding the image obtaining function 111, the processing function 112, the corrected-image generation function 113, and the display control function 114; the detailed explanation is given later.

The input interface 12 is configured using an input device that receives various input operations from the user. The input interface 12 receives an input operation from the user, and outputs an electrical signal corresponding to the received input operation to the processing circuitry 11. For example, the input interface 12 is configured using a mouse, or a keyboard, or various buttons such as a trackball, or a touchpad in which an input operation is performed by touching the operation screen, or a touchscreen in which a display screen and a touchpad are integrated, or a contactless input circuit in which an optical sensor is used, or a voice input circuit. The input interface 12 can also be configured using a tablet terminal capable of performing wireless communication with the device main body. Meanwhile, the input interface 12 is not limited to include a physical operation component such as a mouse or a keyboard. That is, examples of the input interface 12 also include an electrical signal processing circuit that receives an electrical signal corresponding to an input operation from an external input device installed separately from the device, and that outputs the electrical signal to the processing circuitry 11.

The display 13 is configured using a display device that displays a variety of information. For example, the display 13 displays a graphical user interface (GUI) and various X-ray images under the control of the processing circuitry 11.

The memory circuitry 14 is configured using, for example, a semiconductor memory device such as a random access memory (RAM) or a flash memory; or a hard disk; or an optical disk. The memory circuitry 14 is used to store a variety of data and various computer programs. For example, the memory circuitry 14 is used to store a graphical user interface (GUI), X-ray images, and X-ray image data. Moreover, the memory circuitry 14 is used to store a computer program that is executed by the processing circuitry 11 and that causes the processing circuitry 11 to function as the image obtaining function 111, the processing function 112, the corrected-image generation function 113, and the display control function 114.

Till now, the explanation was given about an exemplary configuration of the medical image processing apparatus 1 according to the first embodiment. With such a configuration, the medical image processing apparatus 1 according to the first embodiment enables achieving enhancement in the visibility of a device captured in an X-ray image. More particularly, the medical image processing apparatus 1 performs registration of the device based on a plurality of X-ray images collected during such a period of time within which the device in the X-ray images undergoes fewer changes attributed to the cardiac pulsation of the subject. As a result, in the medical image processing apparatus 1, registration of the device is performed without experiencing an impact of the cardiac pulsation. That enables achieving enhancement in the visibility of the device.

As explained above, in the vascular intervention treatment, the medical image processing apparatus becomes able to perform video display in which the device virtually appears to be still. Moreover, the medical image processing apparatus can also perform an addition operation with respect to a plurality of X-ray images that is position-adjusted using the markers as the guide, and can display the device in a highlighted manner. For example, in the treatment for coronary stenosis, the doctor observes fluoroscopic X-ray images and decides on the position for placing a stent. However, the stent has lower radio-opacity than the radio-opacity of the markers, and has poor visibility. In that regard, in the medical image processing apparatus, a plurality of fluoroscopic X-ray images is position-adjusted using the markers as the guide, and then X-ray images are added so as to highlight the stent. That provides support to the placement of the stent.

However, in the medical image processing apparatus, at the time of highlighting the device, if the addition operation is performed without differentiating between the images having large motion blur of the device due to the cardiac pulsation and the images having small motion blur of the device, then the target device for highlighting gets visualized in an obscure manner.

Moreover, if there is any modification in the shape of the device accompanying tortuousness of the veins attributed to the cardiac pulsation, sometimes the medical image processing apparatus is not able to perform registration of the device with accuracy. For example, the medical image processing apparatus sequentially obtains the images of a plurality of collected frames; performs image deformation and rotation using the two markers, which are detected from the image of the previous frame in chronological order, as the guide; and performs the registration of the device. At that time, although the registration is performed with respect to the markers among the frames, the shapes of the device existing between the two markers are not position-adjusted. For that reason, if the obtained X-ray images of a plurality of frames include images having large modification in the shape of the device due to the cardiac pulsation and images having small modification in the shape of the device; then, of the stent or the balloon visualized in the X-ray images, in the portion other than the surrounding portion of the markers, some part may get displayed in an obscure manner.

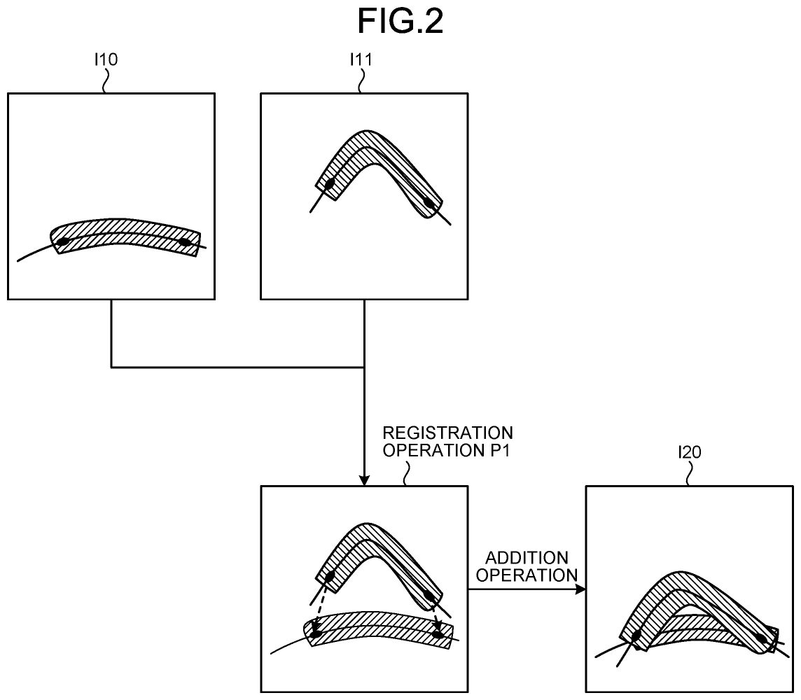

FIG. 2 is a diagram illustrating an example of a registration operation and an addition operation performed with respect to the X-ray images in which a stent is visualized. With reference to FIG. 2, the explanation is given about an overview of a registration operation P1 and an addition operation performed regarding the stent that is bent due to cardiac pulsation. In a medical image processing apparatus, the registration operation P1 is performed to obtain, from among the sets of X-ray image data sequentially collected in chronological order, the latest set of X-ray image data and a predetermined number of sets of X-ray image data collected before that. For example, as illustrated in FIG. 2, X-ray image data I11 representing the latest data is obtained along with X-ray image data I10 that was collected earlier. Then, based on the positions of the two markers in the X-ray image data I10, the registration operation P1 is performed so as to adjust the positions of the markers in the X-ray image data I11. Herein, there are times when the strut, which is the metallic mesh in the stent, changes its shape due to the tortuousness of the veins attributed to the cardiac pulsation. That results in a change in the mesh design of the strut.

However, as illustrated in FIG. 2, the registration operation P1 is meant to perform image deformation only with respect to the markers. Thus, image deformation is not performed with respect to the other portion other than the markers. Then, in the addition operation performed after the registration operation P1, the X-ray image data I11 including the deformed strut is added to the X-ray image data I10. As a result, in a post-added image 120 that is generated, the shape of the stent or the mesh design of the stent becomes obscure, thereby causing a decline in the visibility of the stent for the user.

In that regard, in the medical image processing apparatus 1 according to the first embodiment, a characteristic region of the device is identified in such X-ray images which, based on electrocardiogram information, are collected during the period of time within which there is less movement attributed to the cardiac pulsation; and then the registration is performed based on the characteristic region of the device. As a result, the medical image processing apparatus 1 displays images in which the registration of the device is performed among the frames while holding down the impact of the cardiac pulsation.

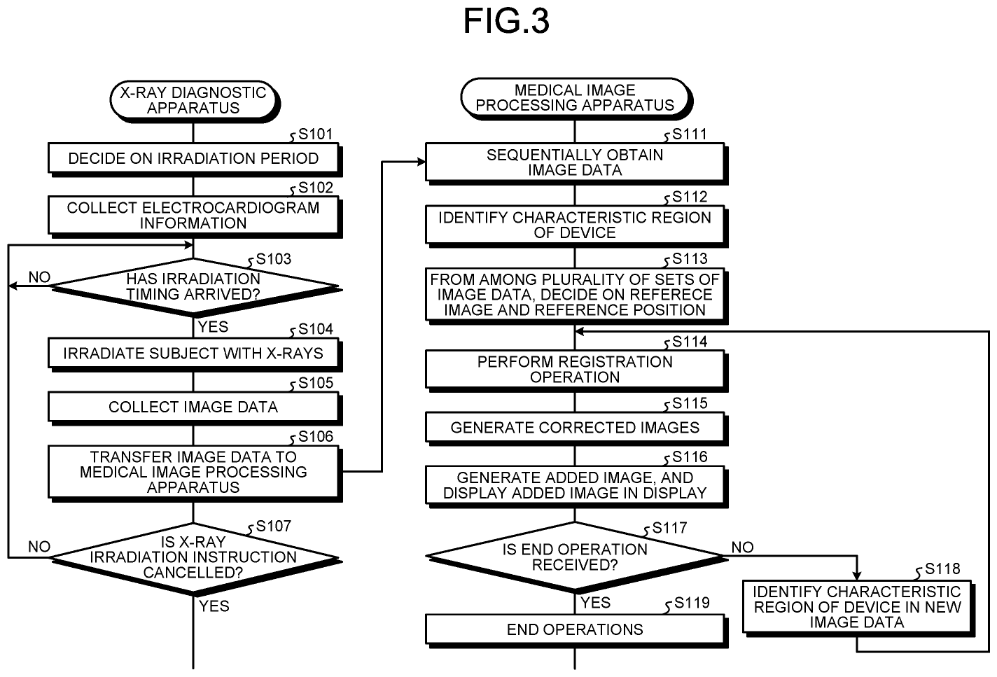

Given below is the explanation of the sequence of operations performed in the medical image processing apparatus 1. FIG. 3 is a sequence diagram illustrating the sequence of operations performed in the medical image processing apparatus 1 and the sequence of operations performed in the X-ray diagnostic apparatus 2 according to the first embodiment. In the following explanation, firstly, the overview of the steps in the sequence diagram is given, and that is followed by the detailed explanation of the operation performed at each step. The operations from Step S101 to Step S107 illustrated in FIG. 3 are performed by the processing circuitry 27 of the X-ray diagnostic apparatus 2. The operation at Step S111 is performed when the processing circuitry 11 reads the computer program corresponding to the image obtaining function 111 from the memory circuitry 14 and executes the computer program. The operations from Step S112 to Step S114 are performed when the processing circuitry 11 reads the computer program corresponding to the processing function 112 from the memory circuitry 14 and executes the computer program. The operation at Step S115 is performed when the processing circuitry 11 reads the computer programs corresponding to the corrected-image generation function 113 and the display control function 114 from the memory circuitry 14 and executes the computer programs. The operation at Step S116 is performed when the processing circuitry 11 reads the computer program corresponding to the display control function 114 from the memory circuitry 14 and executes the computer program. The operations from Step S117 to Step S119 are performed when the processing circuitry 11 reads the computer program corresponding to the processing function 112 from the memory circuitry 14 and executes the computer program.

As illustrated in FIG. 3, the processing circuitry 27 of the X-ray diagnostic apparatus 2 according to the first embodiment decides on the irradiation period for irradiating the subject with X-rays (Step S101) and collects electrocardiogram information (Step S102). Then, based on the electrocardiogram information and based on an X-ray irradiation instruction received via an input interface (for example, pressing of a switch for X-ray irradiation), the processing circuitry 27 determines whether or not the irradiation timing has arrived (Step S103). If the irradiation timing has not yet arrived (No at Step S103), then the system control returns to Step S103. When the irradiation timing arrives (Yes at Step S103), the processing circuitry 27 irradiates the subject with X-rays (Step S104). Subsequently, the processing circuitry 27 collects X-ray image data (Step S105), and transfers it to the medical image processing apparatus 1 (Step S106). Then, the processing circuitry 27 determines whether or not an X-ray irradiation instruction is cancelled via the input interface (Step S107). If the X-ray irradiation instruction has not been cancelled (No at Step S107), then the system control returns to Step S103. When the X-ray irradiation instruction is cancelled (Yes at Step S107), the processing circuitry 27 ends the operations performed in the X-ray diagnostic apparatus 2.

After the transfer of the X-ray image data at Step S106, in the medical image processing apparatus 1 according to the first embodiment, the processing circuitry 11 sequentially obtains the sets of X-ray image data (Step S111). Then, the processing circuitry 11 identifies the characteristic region of the device in the sets of X-ray image data (Step S112) and decides on a reference image and a reference position (Step S113). Subsequently, the processing circuitry 11 performs the registration operation based on the reference position (Step S114) and generates corrected images (Step S115). Moreover, the processing circuitry 11 generates added-image data based on the corrected images, and displays the added-image data in a display (Step S116). Then, the processing circuitry 11 determines whether or not an end instruction is received from the operator via the input interface 12 as an instruction for ending the operations (Step S117). If the end operation is not yet received (No at Step S117), then the processing circuitry 11 identifies the characteristic region of the device in a newly-obtained set of image data (Step S118) and performs the registration operation. When an end instruction is received (Yes at Step S117), the processing circuitry 11 ends the operations performed in the medical image processing apparatus 1 (Step S119).

Given below is the detailed explanation of the operations performed in the medical image processing apparatus 1 and the X-ray diagnostic apparatus 2 according to the first embodiment. The operator presses a foot switch and issues an X-ray irradiation instruction. In the X-ray diagnostic apparatus, when the input interface receives the X-ray irradiation instruction, the processing circuitry 27 emits X-rays only for a partial period of time during a cardiac phase of the subject based on electrocardiogram information and collects X-ray image data. When the input interface no more receives an X-ray irradiation instruction, the X-ray diagnostic apparatus 2 ends X-ray irradiation. Herein, the partial period of time during a cardiac phase includes the period of time in which there is relatively less movement in the cardiac pulsation of the heart of the subject. Moreover, the period of time having relatively less movement is equivalent to the period of time in which there is relatively less fluctuation in the electrocardiographic complex of a single cardiac beat. For example, the period of time having relatively less movement is equivalent to the period of time in which there is less movement of the heart during ventricular systole and the period of time in which there is less movement of the heart during ventricular diastole.

Meanwhile, during a single cardiac beat, the heart undergoes contraction and expansion so as to pump blood out of the heart. More particularly, during a single cardiac beat, the atrium contracts (P wave in an electrocardiographic complex) and then the ventricle contracts ((Q wave)˜(R wave)˜(S wave)). Subsequently, the ventricle expands (T wave). At that time, the ventricular systole is equivalent to the period of time from the peak of the R wave to the vicinity of the end of the T wave, and the ventricular diastole is equivalent to the period of time from the vicinity of the end of the T wave to the peak of the next R wave. Although the heart performs a significant movement during contraction and expansion, the movement is relatively less during a partial period of time of the ventricular systole and a partial period of time of the ventricular diastole. In that regard, in the first embodiment, the X-ray image data is collected in the period of time in which the heart movement is less during at least either the ventricular systole or the ventricular diastole.

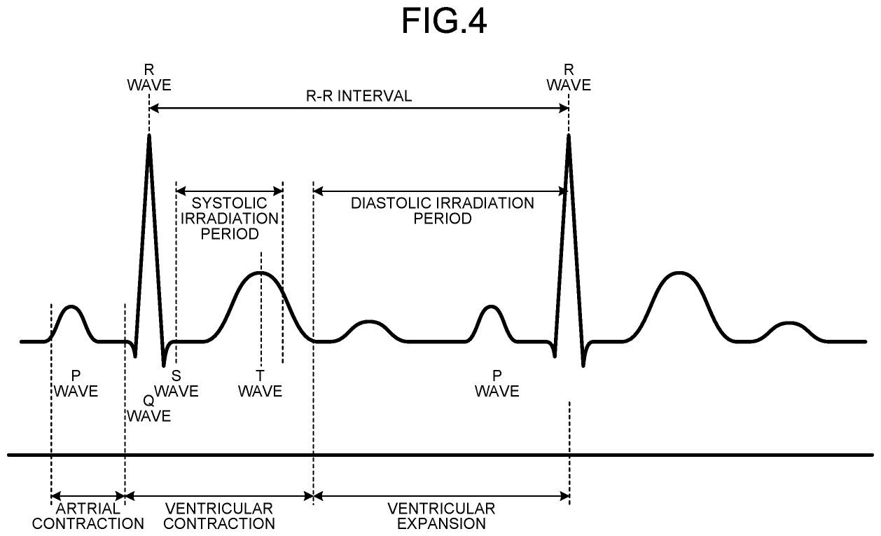

In the X-ray diagnostic apparatus 2, based on the electrocardiogram information, an image collection function of the processing circuitry 27 decides on the irradiation period in which the subject is to be irradiated with X-rays in a continuous manner for a plurality of number of times during a single cardiac beat. For example, based on the electrocardiogram information obtained by the X-ray diagnostic apparatus 2 from an electrocardiograph, the image collection function decides that at least either a systolic phase irradiation period illustrated in FIG. 4 or a diastolic phase irradiation period illustrated in FIG. 4 represents the irradiation period. Meanwhile, as far as deciding on at least either the systolic phase irradiation period or the diastolic phase irradiation period as the irradiation period is concerned, the image collection function can make the decision based on the specification given by the operator via the input interface, or can make the decision based on preset information. The systolic phase irradiation period represents the period of time in which there is less movement of the heart during the ventricular systole. In an identical manner, the diastolic phase irradiation period represents the period of time in which there is less movement of the heart during the ventricular diastole. FIG. 4 is a diagram illustrating an example of the correspondence relationship between the electrocardiogram and the periodic movement of the heart according to the first embodiment.

The electrocardiogram information represents information that, regarding an electrocardiogram (ECG) collected from the subject in real time, contains the timings of generation of the R wave and the T wave as identified by an electrocardiograph. For example, the electrocardiogram information represents data in which the electrocardiogram is recorded along with the timings of generation of the R wave and the T wave (i.e., timestamp information). Meanwhile, the electrocardiogram information need not always represent the information about the detection of the R wave and the T wave. Alternatively, for example, the electrocardiogram information can be information about the detection of only the R wave. Moreover, the electrocardiogram information is not limited to the information about the R wave and the T wave, and alternatively can be information about the detection of the timings of generation of the P wave, the Q wave, the R wave, and the S wave.

The image collection function reads, from preset information that is set in advance, and sets the following information: a start point S of the irradiation period that has been decided; an end point E of the irradiation period; and the X-ray irradiation interval. The X-ray irradiation interval represents the pulse rate of the continuous X-ray irradiation performed between the start point S of the irradiation period and the end point E of the irradiation period. For example, the image collection function reads the pulse rate from X-ray conditions set in advance, and accordingly decides on the X-ray irradiation interval. Herein, it is possible to have a different pulse rate set for each irradiation period.

As far as the preset information is concerned; the R-R interval of the subject, the ventricular systole, and the ventricular diastole are hypothesized from the standard tendency of a cardiac phase of a human body, and the preset information is set with reference to that tendency of the cardiac phase. In other words, the preset information contains the start point S of the irradiation period and the end point E of the irradiation period that are decided based on the possible heart-rate variability values in a human body, such as the standard R-R interval or the occurrence rate of the ventricular systole and the ventricular diastole in a single cardiac beat.

However, the tendency of the cardiac phases is different for each subject. For example, depending on the subject, in a single cardiac beat, there are times when the systolic phase irradiation period has a higher proportion as compared to the preset information and the diastolic phase irradiation period has a lower proportion as compared to the preset information. Moreover, there is a possibility that the R-R interval of the subject is longer by α % as compared to the R-R interval in the preset information. In that regard, the image collection function can compare the irradiation period read from the preset information with the actual electrocardiogram information, and accordingly adjust the start point S of the irradiation period and the end point E of the irradiation period. More particularly, based on the electrocardiogram information and the recent condition of the subject, the image collection function estimates the period of time having less movement of the heart, and adjusts the start point S of the irradiation period and the end point E of the irradiation period of the preset information in such a way that the irradiation period becomes equal to the estimated period of time. Moreover, along with adjusting the irradiation period, the image collection function can also increase or decrease the X-ray irradiation count.

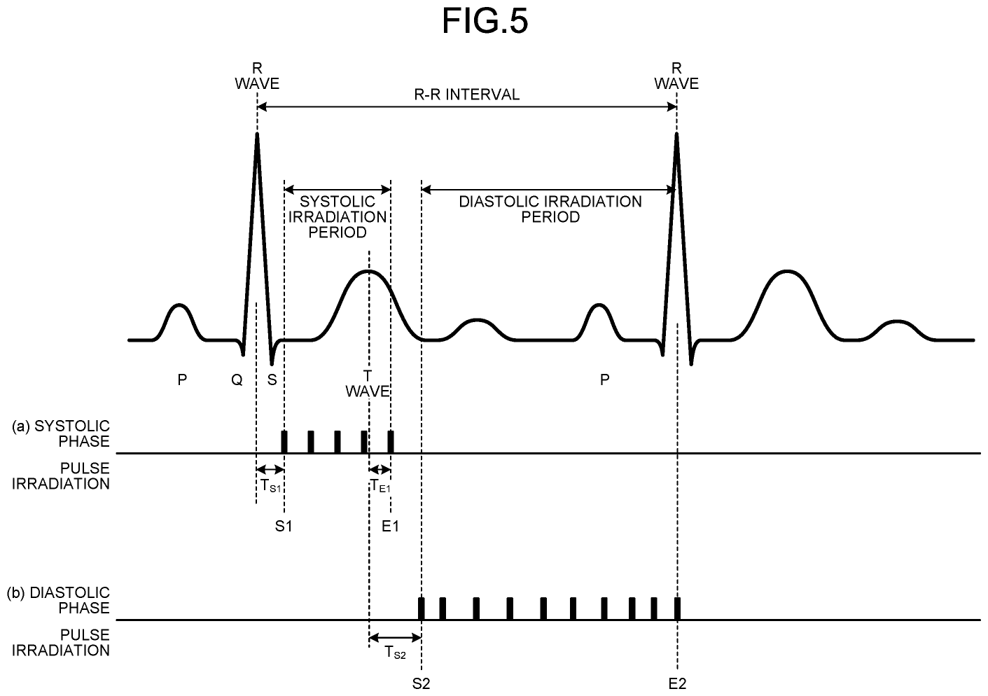

FIG. 5 is a diagram illustrating an example of the correspondence relationship between the irradiation period and the pulse irradiation according to the first embodiment. In FIG. 5 is illustrated the correspondence relationship between the electrocardiogram, two types of irradiation periods, and the pulse irradiation corresponding to each irradiation period. In FIG. 5, (a) systolic pulse irradiation represents the pulse irradiation corresponding to the systolic phase irradiation period. The image collection function decides on a start point S1 of the irradiation period equivalent to the first instance of pulse irradiation during the systolic phase irradiation period; decides on an end point E1 of the irradiation period equivalent to the last instance of pulse irradiation during the systolic phase irradiation period; and decides on the irradiation interval. For example, in the example illustrated in FIG. 5, based on the preset information, it is decided that the start point S1 of the irradiation period arrives after the elapse of T_{S1} seconds since the detection of the previous R wave according to the electrocardiogram information. Moreover, based on the preset information, it is decided that the end point E1 of the irradiation period arrives after the elapse of T_{E1} seconds since the detection of the T wave. Furthermore, the image collection function reads, from the preset information, and sets the irradiation interval during the systolic phase irradiation period; and decides on the X-ray irradiation count between the start point S1 of the irradiation period and the end point E1 of the irradiation period. In FIG. 5 is illustrated the case in which X-ray irradiation is performed for five times during the systolic phase irradiation period. The image collection function can compare the preset information with the recent condition of the subject or with the electrocardiogram information, and accordingly increase or decrease the irradiation count.

In an identical manner, in FIG. 5, (b) diastolic pulse irradiation represents the pulse irradiation corresponding to the diastolic phase irradiation period. The image collection function decides on a start point S2 of the irradiation period equivalent to the first instance of pulse irradiation during the diastolic phase irradiation period; decides on an end point E2 of the irradiation period equivalent to the last instance of pulse irradiation during the diastolic phase irradiation period; and decides on the irradiation interval. For example, in the example illustrated in FIG. 5, based on the preset information, it is decided that the start point S2 of the irradiation period arrives after the elapse of T_{S2} seconds since the detection of the previous T wave according to the electrocardiogram information. Moreover, based on the preset information, it is decided that the end point E2 of the irradiation period represents the subsequently-detected R wave. The irradiation interval during the diastolic irradiation period is set by reading it from the preset information. In FIG. 5 is illustrated the case in which X-ray irradiation is performed for 10 times during the diastolic phase irradiation period. The image collection function can refer to the recent condition of the subject, the electrocardiogram information, and the preset information; and can accordingly change the irradiation count.

Meanwhile, the preset information about the start point and the end point of each irradiation period can be set during the period of time that goes by after the detection of the R wave by the electrocardiograph. For example, regarding (a) systolic pulse irradiation, the start point S1 of the irradiation period can be set to arrive after the elapse of T_{S1} seconds since the detection of the R wave, and the end point E1 of the irradiation period can be set to arrive after the elapse of T_{E1-R} seconds since the detection of the R wave. In an identical manner, regarding (b) diastolic pulse irradiation, the start point S2 of the irradiation period can be set to arrive after the elapse of T_{S2-R} seconds since the detection of the R wave, and the end point E2 of the irradiation period can be set to arrive after the elapse of T_{E2-R} seconds since the detection of the R wave.

Moreover, the preset information about the start point and the end point of each irradiation period can be set based on the period of time that is generally considered convenient for collection, such as the R-R interval indicating the period of time between the R waves. More particularly, the timing of generation of the previous R wave is treated as 0% of the R-R interval, and the timing of generation of the next R wave is treated as 100% of the R-R interval. In that case, for example, in (a) systolic pulse irradiation, the start point S1 during the irradiation period can be set at 7% of the R-R interval, and the end point E1 of the irradiation period can be set at 28% of the R-R interval. In an identical manner, regarding (b) diastolic pulse irradiation, the start point S2 of the irradiation period is set at 35% of the R-R interval, and the end point E2 of the irradiation period is set at 100% of the R-R interval. As a result, the image collection function can collect the timing of generation of the previous R wave and the R-R interval from the electrocardiogram information, and can accordingly decide on the X-ray irradiation period.

Meanwhile, in the image collection function, the explanation is given about deciding on either the systolic irradiation period or the diastolic irradiation period as the irradiation period. However, the first embodiment is not limited to that case. Alternatively, the systolic irradiation period as well as the diastolic irradiation period can be decided as the irradiation period. Still alternatively, the irradiation period can be made of a combination of a partial period of the systolic irradiation period and a partial period of the diastolic irradiation period. For example, there are times when the shape of the device undergoes only a small amount of change in the systolic irradiation period and in the diastolic irradiation period. In that regard, based on the electrocardiogram information and a preset threshold value, the pulsation-induced amount of movement of the device in the systolic irradiation period and the diastolic irradiation period is predicted. Then, based on the prediction result, the electrocardiogram information, and the irradiation interval; the image collection function selects a partial period of the systolic irradiation period and a partial period of the diastolic irradiation period, and sets the selected combination as the irradiation period. For example, the image collection function combines the period of time from the start point of the systolic irradiation period till the generation of the T wave with the period of time from the start point of the diastolic irradiation period to the end point of the diastolic irradiation period, and treats combined period of time as the irradiation period. Meanwhile, the method for predicting the amount of movement of the device is not limited to the case of using the electrocardiogram information as explained above. Alternatively, various other methods for predicting the amount of movement of the device can be implemented, such as collecting the X-ray images in advance and calculating the amount of movement of the device in advance.

Then, as illustrated in FIG. 3, the image collection function collects the electrocardiogram information collected in real time by an electrocardiograph. Subsequently, the image collection function determines whether or not the timing is within the decided irradiation period and determines whether or not the input interface of the X-ray diagnostic apparatus 2 has received an X-ray irradiation instruction from the operator. With that, the image collection function determines whether or not the irradiation timing has arrived. For example, at the timing of performing the abovementioned determination, if the timing is within the decided irradiation period and if an X-ray irradiation instruction is received, then the image collection function controls the X-ray tube and the X-ray detector and performs X-ray irradiation. Moreover, at the timing of performing the abovementioned determination, if the timing is outside the decided irradiation period but if an X-ray irradiation instruction is received, then the image collection function controls the X-ray tube and the X-ray detector and performs X-ray irradiation in the next irradiation period. Then, the image collection function collects X-ray image data based on the X-rays detected by the X-ray detector. Moreover, the image collection function transfers the collected X-ray image data to the medical image processing apparatus 1 via a network. Meanwhile, if an X-ray irradiation instruction is not received, then the image collection function does not perform X-ray irradiation even if the timing is within the irradiation period.

For example, in the case of performing irradiation during the diastolic irradiation period, the image collection function continuously refers to the electrocardiogram information and, if an X-ray irradiation instruction is received at a timing not within the diastolic irradiation period, refers to the electrocardiogram information until the detection of the T wave representing the trigger for the start point S2 of the irradiation period. Once the occurrence of the T wave is detected, the image collection function irradiates the subject with X-rays after the elapse of T_{S2} seconds since the detection of the T wave (i.e., at the start point S2 of the irradiation period). Then, the image collection function performs pulse irradiation at the set irradiation interval A [fps] until the occurrence of the next R wave (the end point E2 of the irradiation period). During that period of time, the image collection function sequentially collects and transfers the X-ray image data.

The image obtaining function 111 obtains the sets of X-ray image data that are sequentially collected during a partial period of time in the cardiac phase of the subject who has a device inserted in the body. For example, the image obtaining function 111 sequentially obtains the sets of X-ray image data from the X-ray diagnostic apparatus 2 via a network. With that, the image obtaining function 111 obtains the sets of X-ray image data which are collected in the period of time having less movement of the heart and in which the device is visualized.

The processing function 112 identifies the characteristic region of the device captured in each of a plurality of sets of X-ray image data. More particularly, the processing function 112 detects at least a single characteristic object of the device that is visualized in a plurality of sets of X-ray image data; and identifies, as the characteristic region in each set of X-ray image data, the region in which the concerned characteristic object of the device is captured. Herein, a characteristic object of the device represents an object having relatively high X-ray opacity in the device, such as a marker visualized in an X-ray image. The method for detecting a characteristic object can be a known method, such as the method for template matching using a teacher image or the method for characteristic object detection based on the generation of a high-frequency image including the high-frequency component of the X-ray image data. Alternatively, a characteristic object can be detected when the operator specifies the characteristic object in at least one set of X-ray image data via the input interface 12. Meanwhile, as long as a single characteristic object is present, it serves the purpose.

As far as the identification of the characteristic region of the device is concerned, either one of the following can be implemented: (1) based on the position of the detected characteristic object, a specific part of the concerned device is identified in the X-ray images and the region of that specific part or the neighborhood thereof can be treated as the characteristic region; or (2) the region of the detected characteristic object or the neighborhood thereof can be treated as the characteristic region. Consider a case in which a guide wire and a stent having two markers are visualized in a plurality of sets of obtained X-ray image data. With reference to (1) mentioned above, the processing function 112 detects the two markers, which represent the characteristic objects of the device, in each set of X-ray image data. Then, based on the detected positions of the two markers, the processing function 112 detects the guide wire that joins the two markers in the X-ray image data and identifies, as the characteristic region, either the region in which the two markers and the guide wire are visualized or the neighborhood of that region. As a result, the processing function 112 identifies the characteristic region that includes the markers representing the characteristic objects. Regarding the same case, with reference to (2) mentioned above, the processing function 112 detects the two markers representing the characteristic objects in each X-ray image data. Then, the processing function 112 identifies, as the characteristic region, either the region in which both markers are visualized or the neighborhood of that region. As a result, the processing function 112 identifies the characteristic region that includes the markers representing the characteristic objects.

Then, the processing function 112 sets, as reference image data for a registration operation P2, one of a plurality of sets of X-ray image data. Subsequently, the processing function 112 sets, as the reference position, the position of the characteristic region in the reference image data. More particularly, the processing function 112 sets, as the reference image data, the X-ray image data which, from among a plurality of sets of X-ray image data that is obtained, is initially collected in a partial period of time in a cardiac phase of the subject. For example, if a plurality of sets of X-ray image data is obtained as a result of sequential irradiation from the start point S2 of the diastolic irradiation period; then the processing function 112 sets, as the reference image data, the set of X-ray image data collected corresponding to the irradiation at the start point S2 of the irradiation period. Moreover, for example, the processing function 112 sets, as the reference position, the position of the characteristic region of the device, that is, the position of the region that includes the two markers and that is detected from the reference image data.

Meanwhile, the reference image data set by the processing function 112 is not limited to the set of X-ray image data that is initially collected during the irradiation period. Alternatively, for example, the processing function 112 sets, as the reference image data, the image decided according to an operation performed by the operator. More particularly, in response to an X-ray irradiation instruction issued by the operator (for example, in response to the pressing the foot switch), the processing function 112 sets, as the reference image data, the X-ray image data that is initially collected after receiving the X-ray irradiation instruction. Still alternatively, from among the sets of X-ray image data collected in response to an X-ray irradiation instruction, the set of X-ray image data collected after N number of frames can be treated as the reference image data. Still alternatively, if the operator has specified the reference image data from among a plurality of sets of X-ray image data, then the processing function 112 can treat the specified X-ray image data as the reference image data.

Still alternatively, the processing function 112 can decide on the reference image data by evaluating the set of X-ray image data. More particularly, based on the shapes of a plurality of characteristic regions of the device, the processing function 112 evaluates the degree of flexion of the shape of each characteristic region. Then, based on the evaluation result, the processing function 112 sets the image having the smallest degree of flexion of the device as the reference image data. Meanwhile, the degree of flexion of a shape indicates the degree of flexion of the device between the markers. For example, the processing function 112 sets, as the degree of flexion, the distance between the two markers as detected from each of a plurality of sets of X-ray image data. Then, the processing function 112 calculates the degrees of flexion (the distance between the two markers) for a plurality of sets of X-ray image data and, if any degree of flexion is greater than a set threshold value, considers that there is no flexion in the concerned device and extracts the corresponding set of X-ray image data. Then, from among the sets of X-ray image data having the degrees of flexion to be greater than the threshold value, the processing function 112 sets the X-ray image data having the earliest collection timing as the reference image data. Meanwhile, the threshold value used in evaluating the degree of flexion is not limited to be a fixed value. Alternatively, the average value of the distances between the markers can be used as the threshold value. At that time, if the difference (deviation) between the calculated degree of flexion and the average value is small, then the processing function 112 evaluates that the device has only a small flexion. More particularly, in the sets of X-ray image data in which the difference between the corresponding calculated distance between the markers and the average value of the distances is small, the processing function 112 evaluates that the device has only a small flexion.

Herein, the method for calculating the degree of flexion regarding the characteristic region including two markers is not limited to the abovementioned calculation method based on the distance between the markers. More particularly, the processing function 112 can calculate the degree of flexion based on the contour of the characteristic region.

Then, based on the reference position, the processing function 112 adjusts the position of the characteristic region in the sets of the X-ray image data collected after the reference image data. More particularly, regarding a plurality of identified characteristic regions of the device, in accordance with the shape of the characteristic region of the device captured in the reference image data, the processing function 112 modifies the shape of the characteristic region of the device captured in the other sets of X-ray image data other than the reference image data. Thus, the processing function 112 performs a shape modification operation (the registration operation P2) for matching the shapes of a plurality of characteristic regions of the device.

For example, the processing function 112 performs rotation, parallel translation, magnification, and reduction with respect to the characteristic regions of a plurality of sets of X-ray image data collected after the reference image data. As a result, the processing function 112 performs registration in such a way that the positions of the two markers captured in the target image data for modification are matched to the positions of the two markers captured in the reference image data.

The corrected-image generation function 113 generates, based on the registration operation, sets of corrected-image data from the sets of X-ray image data collected after the reference image data. For example, the corrected-image generation function 113 generates sets of corrected-image data representing the result of performing the registration operation P2 with respect to a plurality of X-ray image data. Then, the corrected-image generation function 113 stores the generated sets of corrected-image data in the memory circuitry 14.

The display control function 114 displays, in the display 13, added-image data generated by adding a plurality of sets of corrected-image data. More particularly, the display control function 114 displays, in the display 13, added-image data generated by performing weighted-addition of a plurality of sets of corrected-image data. Then, every time a new set of corrected-image data is newly created in chronological order, the display control function 114 creates added-image data using the sets of corrected-image data equal in number to the frame count that is set. For example, if the preset frame count is equal to five, every time a set of corrected-image data is newly generated, the display control function 114 creates added-image data using the sets of corrected-image data of the previous five frames in chronological order that include the newly-generated set of corrected-image data. Then, the display control function 114 displays the added-image data in the display 13. As a result, the image displayed in the display 13 represents a highlighted-device image in which the latest shape of the device is visualized.

Meanwhile, the display control function 114 can involve the reference image data too in the addition operation. Moreover, the weighting coefficient used in the addition operation can be set using the preset information. Furthermore, the method for setting the weighting coefficient can be a known method. The method for performing the addition operation is not limited to adding the sets of corrected-image data equal in number to the frame count that is set. Alternatively, a new set of corrected-image data can be added to the added-image data created in the past. For example, the display control function 114 can create added-image data by adding a newly-created set of corrected-image data, which is newly created in chronological order, to the previously-created added-image data.

With such a configuration, in the medical image processing apparatus 1, based on the electrocardiogram information, such sets of X-ray image data are used which are collected in the period of time having less movement attributed to the pulsation. As a result, the sets of X-ray image data in which there is less deformation of the device can be treated as the processing targets. As a result, in the medical image processing apparatus 1, also in the case when a registration operation, such as the registration operation P1, is performed in which the region of the markers or the neighborhood thereof is treated as the characteristic region (for example, the registration operation in which only the two markers serve as the guide), it still becomes possible to generate images in which there is high visibility of the device.

As explained above, in the medical image processing apparatus 1, the sets of X-ray image data that are collected in the period of time having less pulsation-induced movement are treated as the targets for the registration operation. That enables achieving enhancement in the visibility of the device. However, there are also times when the sets of X-ray image data that are collected in the period of time having less pulsation-induced movement include the sets of X-ray image data in which the device, such as a stent, has a flexion formed therein. In that regard, in the medical image processing apparatus 1, the registration operation can be performed also by taking into account the detailed shape of the characteristic region of the device. Given below is the explanation about the case of visualizing two markers in a plurality of sets of X-ray image data and visualizing a guide wire and a stent having the markers.

The processing function 112 detects, in each of a plurality of sets of X-ray image data, two markers that represent the characteristic objects of the device. Then, in the X-ray image data, using the two detected markers as the guide, the processing function 112 detects the guide wire present between the markers. Subsequently, the processing function 112 treats the region including the two markers and the detected guide wire as the characteristic region of the device. As far as the method for detecting the guide wire is concerned, a known method can be implemented. For example, the guide wire can be detected by performing image processing to search for high X-ray opacity in the region in the vicinity of the markers. Meanwhile, instead of detecting only the markers and the guide wire, the processing function 112 can detect the region including the markers, the guide wire, and the stent as the characteristic region of the device.

Then, from among the plurality of sets of X-ray image data that is obtained, the processing function 112 sets the reference image data to be used in the registration operation P2. As explained earlier, the reference image data can be the initially-collected set of X-ray image data in a partial period of time in the cardiac phase of the subject, or can be the set of X-ray image data decided according to the operation performed by the operator.

Moreover, as explained earlier, based on the shapes of a plurality of characteristic regions of the device, the processing function 112 can evaluate the degree of flexion of each shape of the characteristic region of the device and can set, as the reference image data, the image having the least flexion of the device based on the evaluation result. Herein, an identified characteristic region of the device has the shape formed along the guide wire present between the markers. Based on that, the processing function 112 calculates the degree of flexion of the guide wire in the characteristic region of the device, and accordingly calculates the degree of flexion of the shape of the characteristic region. Alternatively, the processing function 112 can calculate the degree of flexion of the shape of the characteristic region of the device based on the contour of the characteristic region of the device.

Subsequently, the processing function 112 compares the calculated degree of flexion of each guide wire with a threshold value set in advance, and sets the set of X-ray image data having the least flexion of the guide wire as the reference image data. Meanwhile, the threshold value used in evaluating the degree of flexion is not limited to be a fixed value. Alternatively, the average value of the degrees of flexion of the guide wires can be used as the threshold value. If the difference (deviation) between a calculated degree of flexion and the average value is small, then the processing function 112 evaluates that the device has only a small flexion. Herein, the method for calculating the degree of flexion of the shape of the characteristic region is not limited to calculating the degree of flexion of the guide wire as explained above. Alternatively, the degree of flexion can be calculated from the distance between the two markers. Still alternatively, the degree of flexion of the shape of the characteristic region can be calculated according to the combination of the degree of flexion of the guide wire and the distance between the two markers.

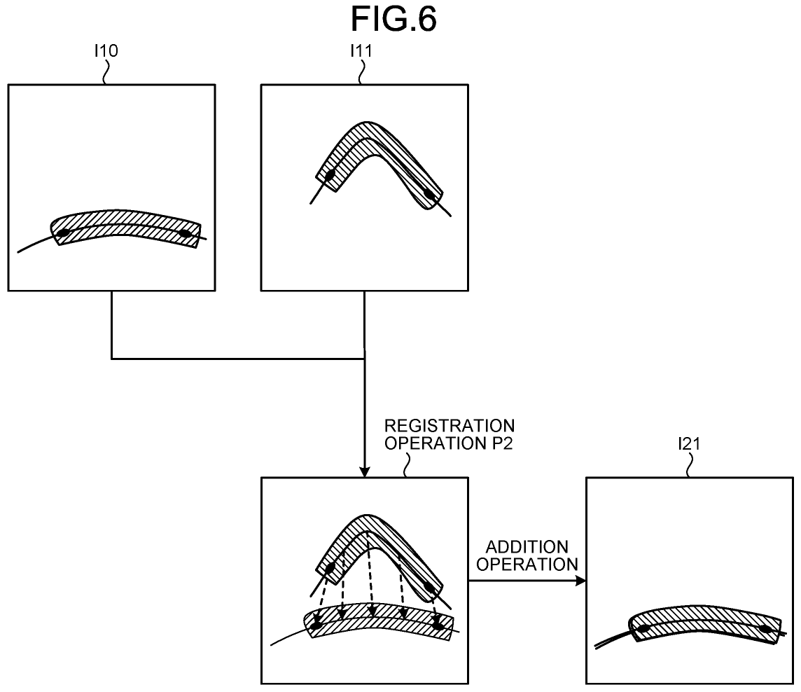

FIG. 6 is a diagram illustrating an example of the registration operation and the addition operation performed according to the first embodiment. Explained below with reference to FIG. 6 is the overview of the registration operation P2 and the addition operation performed regarding a stent having a flexion formed therein due to the pulsation. For example, as illustrated in FIG. 6, the processing function 112 sets the X-ray image data 110 as reference image data I10 from among a plurality of sets of X-ray image data. The X-ray image data I11 represents the set of X-ray image data collected after the X-ray image data 110. With reference to the shape of the characteristic region including the two markers and the guide wire in the reference image data I10, the processing function 112 performs the registration operation P2 for modifying the shape of the characteristic region including the two markers and the guide wire in the X-ray image data I11. Then, the display control function 114 performs the addition operation using the corrected-image data generated after performing the registration operation P2, and generates and displays X-ray image data 121. In the first embodiment, since the registration is performed for modifying the shape of the characteristic region, the X-ray image data 121 that is generated as a result of the addition operation enables achieving enhancement in the visibility of the device as illustrated in FIG. 6. Given below is the detailed explanation of the operations.

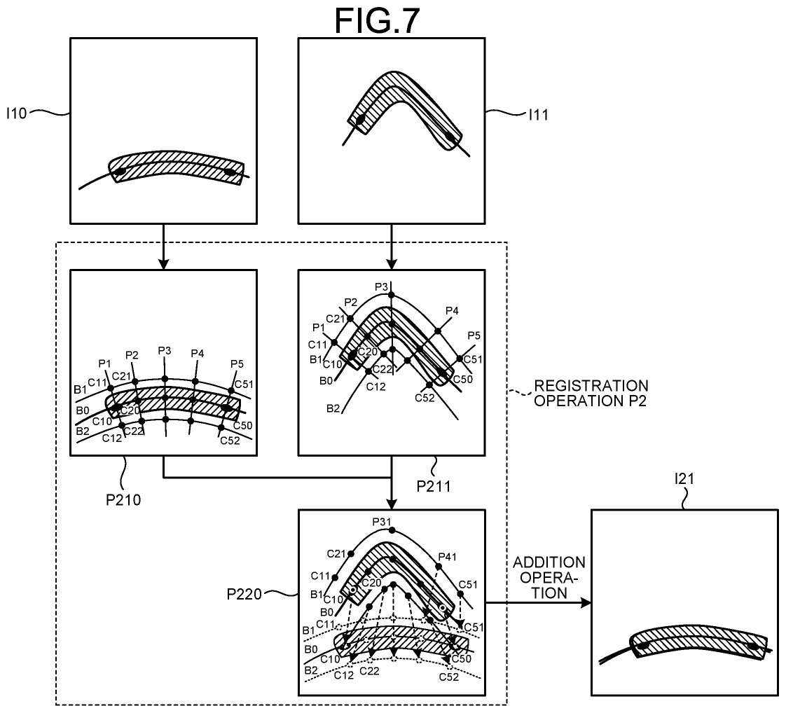

FIG. 7 is a diagram illustrating an example of the details of the registration operation performed according to the first embodiment. In FIG. 7, the explanation is given about the registration operation P2 that includes a registration operation P210 performed with respect to the reference image data I10, a registration operation P211 performed with respect to the X-ray image data I11, and a registration operation P220; and the explanation is given about an example of the X-ray image data 121 generated as a result of performing the addition operation after the registration operation P2.

The processing function 112 sets the positions of a plurality of characteristic points in a set of X-ray image data according to the position of the characteristic object present in the characteristic region of the device and according to the shape of the characteristic region. The processing function 112 sets the positions of the characteristic points in the characteristic regions of a plurality of sets of X-ray image data, so that a plurality of characteristic points reflecting the shape of each characteristic region gets set.

More particularly, the processing function 112 sets the guide wire, which is detected from each set of X-ray image data, as a reference line B0. Moreover, based on the detected positions of the two markers, the processing function 112 sets N number of characteristic points at regular intervals on the segment joining the markers on the reference line B0. Herein, “C” represents the coordinates of a characteristic point. Then, the processing function 112 sets a normal line P of the reference line B0 at each coordinate C. Moreover, the processing function 112 sets a reference line B1 at a distance from the reference line B0 in the positive direction of the normal line P. In an identical manner, the processing function 112 sets a reference line B2 at a distance from the reference line B0 in the negative direction of the normal line P. The reference lines B1 and B2 are assumed to be set at a certain distance from the reference line B0. Then, the processing function 112 sets a characteristic point at the point of intersection between each normal line P and the reference line B1. In an identical manner, the processing function 112 calculates the point of intersection between each normal line P and the reference line B2, and treats the point of intersection as a characteristic point. In other words, the processing function 112 sets N number of characteristic points on the reference line B1 as well as on the reference line B2. In this way, the processing function 112 sets the positions of ((number of reference lines)×N) number of characteristic points, and obtains the shape of the characteristic region including the markers and the guide wire.

For example, in the registration operation P210 illustrated in FIG. 7, based on the positions of the markers detected in the reference image data I10, the processing function 112 sets, at the positions of the two markers, coordinates C10 and C50 as the positions of characteristic points. On the segment joining the coordinates 010 and C50 on the reference line B0, the processing function 112 sets three points (coordinates C20, C30, and C40) at regular intervals. That is, the processing function 112 sets five characteristic points (N=5) on the reference line B0.

Moreover, as the normal lines P of the reference line B0 at the coordinates C, the processing function 112 sets normal lines P1, P2, P3, P4, and P5 at the coordinates 010 to C50, respectively. Then, the processing function 112 sets the reference lines B1 and B2 at a certain distance from the reference line B0. Regarding the normal lines P1 to P5, the processing function 112 calculates coordinates C11, C21, C31, C41, and C51, respectively, that represent the points of intersection with the reference line B1; and treats the coordinates as the characteristic points. Moreover, in an identical manner, the processing function 112 calculates C12, C22, C32, C42, and C52, respectively, that represent the points of intersection with the reference line B2; and treats the coordinates as the characteristic points. With that, the processing function 112 sets five characteristic points on each reference line. Thus, the total number of characteristic points becomes equal to 15.

Then, the processing function 112 sets the same number of characteristic points with respect to the X-ray image data I11. More particularly, as illustrated in FIG. 7, the processing function 112 sets a total of 15 characteristic points as a result of performing the registration operation P211.

Subsequently, regarding the characteristic regions of the other sets of X-ray image data other than the reference image data, the processing function 112 matches the characteristic points to the characteristic points set in the characteristic region of the reference image data. With that, the processing function 112 performs the shape modification operation for modifying the shapes of the characteristic regions of the device, and match the shapes of the characteristic regions of the device among a plurality of sets of X-ray image data. Meanwhile, the shape modification operation can be performed according to a known method regarding non-rigid registration. Moreover, in the shape modification operation, the characteristic points in the other sets of X-ray image data other than the reference image data need not be rigidly matched to the characteristic points in the reference image data. That is, as compared to the positions before performing the shape modification operation, as long as the characteristic points in the other sets of X-ray image data move closer to the characteristic points in the reference image data, it serves the purpose.

More particularly, in the registration operation P220 illustrated in FIG. 7, with reference to the coordinates of the 15 characteristic points set in the reference image data I10, the processing function 112 performs the registration by moving the coordinates of the 15 characteristic points set in the X-ray image data I11 to the corresponding coordinates in the reference image data 110. Accompanying the movement of the characteristic points, the processing function 112 modifies the shape of the characteristic region in each set of X-ray image data. For example, as illustrated in the registration operation P220 in FIG. 7, the coordinates of the characteristic points of the X-ray image data I11 are moved to the coordinates of the corresponding characteristic points in the reference image data I10 that are joined by arrows.

The corrected-image generation function 113 generates a set of X-ray image data that represents the result of performing the registration operation P220, which is illustrated in FIG. 7, regarding the X-ray image data I11; and treats the generated set of X-ray image data as corrected-image data I11. Then, the corrected-image generation function 113 stores the corrected-image data I11 in the memory circuitry 14.

The display control function 114 performs the addition operation using: the corrected-image data I11 generated as a result of performing the registration operation P2 with respect to the X-ray image data I11; set of corrected-image data generated based on other sets of X-ray image data (not illustrated) collected after the reference image data I10; and the reference image data I10. Herein, the X-ray image data 121 illustrated in FIG. 7 represents added-image data 121 created using the reference image data I10 and the corrected-image data I11. The display control function 114 displays the added-image data in the display 13. Every time a new set of added-image data is created, the display control function 114 displays that set of added-image data in the display 13.

As explained above, according to the first embodiment, the image obtaining function 111 obtains X-ray images that are sequentially collected during a partial period of time of the cardiac phase of the subject who has a device inserted in the body. The processing function 112 identifies the characteristic region of the device captured in a plurality of X-ray images; treats the position of the characteristic region identified in the reference image, which is one of a plurality of X-ray images, as the reference position; and, based on the reference position, adjusts the positions of the characteristic regions identified in such X-ray images, from among a plurality of X-ray images, which are collected after the reference image. As a result, in the medical image processing apparatus 1, based on the electrocardiogram information of the subject, X-ray images of a plurality of frames collected in a specific cardiac phase are used. Hence, the X-ray images having reduced inter-frame changes in the device can be treated as the targets for registration of the characteristic region of the device among the frames. As a result, X-ray images having reduced changes in the device can be displayed, thereby enabling achieving enhancement in the visibility of the device in the X-ray images.

Moreover, according to the first embodiment, the image collection function of the X-ray diagnostic apparatus 2 identifies a partial period of time in a cardiac phase based on the electrocardiogram information of the subject, and controls the X-ray tube and the X-ray detector in such a way that X-rays are irradiated during the identified period of time and a plurality of X-ray images is collected. As a result, in the X-ray diagnostic apparatus 2, the X-ray images can be collected during a partial period of time in a cardiac phase based on the electrocardiogram information of the subject. That makes it possible to collect the X-ray images based on the cardiac movement attributed to the pulsation of the subject. As a result, it becomes possible to perform the registration with a higher degree of accuracy, thereby enabling achieving enhancement in the visibility of the device in the X-ray images.

Furthermore, according to the first embodiment, the partial period of time in a cardiac phase is the period of time in which there is relatively less movement in the cardiac pulsation of the subject. As a result, in the medical image processing apparatus 1, it is possible to adjust the position of the device among the X-ray images in which the impact of cardiac pulsation is held down. That makes it possible to provide X-ray images having further reduction in the changes in the device captured therein. That enables achieving enhancement in the visibility of the device captured in the X-ray images.

Moreover, according to the first embodiment, the processing function 112 performs registration according to the shape modification operation in which, regarding a plurality of identified characteristic regions of the device, the shapes of the characteristic regions of the device in a plurality of X-ray images, excluding the reference image, are modified in accordance with the shape of the characteristic region of the device in the reference image that is one of the X-ray images, so that the shapes of the characteristic regions of the device are matched to each other. Thus, in the medical image processing apparatus 1, since the registration operation is performed regarding the shapes of the characteristic regions in a plurality of X-ray images, the accuracy of the registration operation can be enhanced. That enables achieving enhancement in the visibility of the device captured in the X-ray images.

Furthermore, according to the first embodiment, the processing function 112 performs the shape modification operation in which, regarding the characteristic region captured in a plurality of X-ray images, a plurality of characteristic points reflecting the shape of the characteristic region is set; and a plurality of characteristic points set in the characteristic region in the other X-ray images other than the reference image is matched or approximated to a plurality of characteristic points set in the characteristic region in the reference image. Thus, in the medical image processing apparatus 1, since the positions of corresponding characteristic points are adjusted among a plurality of X-ray images, it becomes possible to perform the registration operation that caters to various modifications in the shape and that has high accuracy. That enables achieving enhancement in the visibility of the device in the X-ray images.

Moreover, according to the first embodiment, the processing function 112 detects at least one characteristic object of the device; identifies, as the characteristic region of the device in each X-ray image, the region that includes the characteristic object; and, according to the position of the characteristic object in the characteristic region and according to the shape of the characteristic region, sets the positions of a plurality of characteristic points in the X-ray image. Thus, in the medical image processing apparatus 1, the characteristic region of the device is identified based on a characteristic object, thereby enabling setting the characteristic region in an efficient manner. Moreover, since the positions of the characteristic points are set based on the characteristic object and the shape of the characteristic region, the positions of the characteristic points can be adjusted with high accuracy in a plurality of X-ray images.

Furthermore, according to the first embodiment, based on the registration operation, the corrected-image generation function 113 generates corrected images from the X-ray images that are collected after the reference image. The display control function 114 displays, in the display 13, an added image that is generated by adding a plurality of corrected images. Thus, in the medical image processing apparatus 1, the device that has been accurately position-adjusted is displayed in a highlighted manner, thereby enabling enhancement in the visibility of the device.

First modification example In the first embodiment described above, the explanation is given about the case in which the image obtaining function 111 obtains sets of X-ray image data that are collected after the X-ray irradiation is performed for a plurality of number of times during the irradiation period. However, the first embodiment is not limited to that case. Alternatively, for example, it is possible to think of a case in which, based on the electrocardiogram information, from among a plurality of sets of X-ray image data sequentially generated after the insertion of a device, the image obtaining function 111 obtains a plurality of sets of X-ray image data collected as a result of X-ray irradiation performed during a partial period of time of a cardiac phase.

In that case, in the X-ray diagnostic apparatus 2, when an X-ray irradiation instruction is received from the operator, the X-ray tube and the X-ray detector are controlled and X-ray irradiation is performed for a plurality of number of times at an arbitrary pulse rate. Then, in the X-ray diagnostic apparatus 2, at every instance of collection of the X-rays that have passed through the subject, a set of X-ray image data is generated. Moreover, the X-ray diagnostic apparatus 2 collects each timing of collection of the X-rays that have passed through the subject. At that time, the electrocardiograph that is connected to the X-ray diagnostic apparatus 2 collects the electrocardiogram information of the subject and sends it to the X-ray diagnostic apparatus 2. Then, in the X-ray diagnostic apparatus 2, every time X-ray irradiation is performed, the generated set of X-ray image data, the collection time thereof, and the electrocardiogram information are stored in a corresponding manner in memory circuitry (not illustrated).

Subsequently, from the preset information or via an instruction issued from the input interface 12, the image obtaining function 111 receives the specification of a partial period of time in a cardiac phase of the subject. Accordingly, the image obtaining function 111 obtains, from the memory circuitry of the X-ray diagnostic apparatus 2, the sets of X-ray image data collected during the specified period of time. For example, when an instruction is issued for obtaining the sets of X-ray image data corresponding to the diastolic irradiation period as illustrated in FIG. 4; the image obtaining function 111 reads, from the memory circuitry, and refers to the records in which the sets of X-ray image data, the collection timings thereof, and the electrocardiogram information are held in a corresponding manner; and obtains the sets of X-ray image data collected during to the diastolic irradiation period. Then, in an identical manner to the first embodiment described above, the processing function 112 performs the registration operation and the addition operation with respect to the obtained sets of X-ray image data.

As a result, the X-ray irradiation performed by the X-ray diagnostic apparatus 2 need not be subjected to irradiation control based on the cardiac phase corresponding to the electrocardiographic complex. Then, in the medical image processing apparatus 1, based on the collection timings and the electrocardiogram information associated to the collected sets of X-ray image data, it becomes possible to decide on the sets of X-ray image data to be subjected to the registration operation and the addition operation performed according to the application concerned. As a result, in the medical image processing apparatus 1, display images having high accuracy can be generated using the sets of X-ray image data having less movement attributed to the pulsation. That enables achieving enhancement in the visibility of the device captured in the X-ray images.

Second Modification example

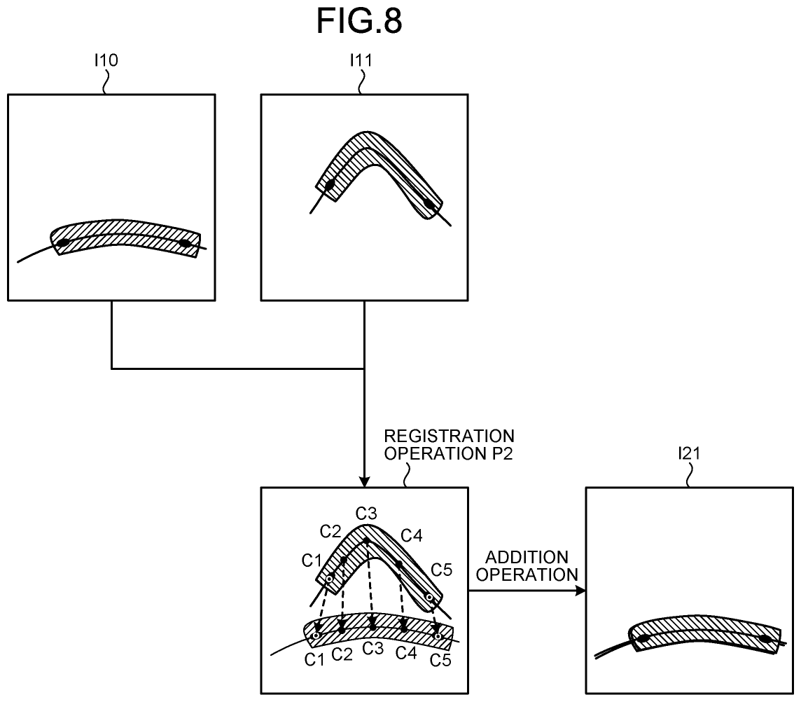

In the first embodiment described earlier, as explained in the registration operation P2 illustrated in FIG. 7, the explanation is given about the case in which, as the method for setting a plurality of characteristic points reflecting the shape of the characteristic region of the device, a guide wire is set as a reference line. Moreover, a plurality of other reference lines is set around the characteristic region, and a plurality of characteristic points is set on each other reference line. However, the first embodiment is not limited to that case. Alternatively, for example, it is also possible to think of case in which the processing function 112 sets a plurality of characteristic points only on the guide wire based on the positions of the markers. FIG. 8 is diagram illustrating an example of the details of the registration operation performed according to the first embodiment. In FIG. 8, the X-ray image data I11 represents the data collected after the reference image data I10. Moreover, in FIG. 8, an example of the registration operation P2 is illustrated that is performed with respect to the reference image data I10, and an example of the X-ray image data 121 is illustrated that is generated as a result of the addition operation performed after the registration operation P2.

Based on the positions of the two markers and the position of the guideline as detected from each set of X-ray image data, the processing function 112 sets N number of characteristic points at regular intervals on the segment joining the markers on the guide wire. Herein, “C” represents the coordinates of a characteristic point. For example, as illustrated in FIG. 8, regarding the reference image data I10 as well as the X-ray image data I11, the processing function 112 sets coordinates C1 and C5 of the characteristic points at the positions of the two detected markers. Then, on the segment joining the coordinates C1 and C5 on the guide line of each set of X-ray image data, the processing function 112 sets three points (coordinates C2, C3, and C4) at regular intervals. That is, the processing function 112 sets five characteristic points (N=5) on the guide wire of each set of image data. Subsequently, the processing function 112 performs the registration in which the coordinates of the characteristic points set in the X-ray image data I11 are moved with reference to the coordinates of the characteristic points set in the reference image data I10.

In the case of performing the registration operation P2 by setting a plurality of characteristic points on the guide wire, it is possible to think of a need to further enhance the accuracy of the registration operation P2. In that case, the processing function 112 evaluates the degree of flexion of the shape of each of a plurality of characteristic regions of the device and, depending on the evaluation result, excludes the shapes of some characteristic regions from the targets for the shape modification operation. More particularly, the processing function 112 obtains the shape of the characteristic region of the device by calculating the radius of curvature of the approximate circle passing through the characteristic points set on the guide wire. Then, the processing function 112 evaluates the degree of flexion, which is calculated for each of a plurality of sets of X-ray image data, using a threshold value set in advance; and excludes such sets of X-ray image data which are evaluated to have a high degree of flexion. Thus, in the medical image processing apparatus 1, since the sets of X-ray image data in which the pulsation-induced movement is smaller than certain conditions, it becomes possible to display such X-ray images in which the movement of the device is further reduced. That enables achieving enhancement in the visibility of the device captured in the X-ray images.

Meanwhile, the threshold value that is set in advance can be of a variety of types. For example, a fixed value of the radius of curvature can be set as the threshold value, or the average of the radii of curvature of the sets of X-ray image data collected in the past can be set as the threshold value. Alternatively, the threshold value can be set to allow an increase of X % with respect to the radius of curvature of the set of X-ray image data collected one frame earlier. In that case, for example, the threshold value becomes equal to ((the curvature of radius of the set of X-ray image data collected one frame earlier)×X/100). Thus, if the radius of curvature of the evaluation target exceeds the threshold value, then the processing function 112 excludes the evaluation target from the target for the shape modification operation. In an identical manner, the threshold value can be set to allow a decrease of Y % with respect to the radius of curvature of the set of X-ray image data collected one frame earlier. In that case, for example, the threshold value becomes equal to ((the curvature of radius of the X-ray image data collected one frame earlier)×(1−Y/100)). Thus, if the radius of curvature of the evaluation target is smaller than the threshold value, then the processing function 112 excludes the evaluation target from the target for the shape modification operation.

Meanwhile, the method for setting a plurality of characteristic points to be used in evaluating the degree of flexion of the shape of the characteristic region is not limited to the method for setting the characteristic points only on the guide wire. More particularly, when a plurality of reference lines is set around the characteristic region as illustrated in FIG. 7, the processing function 112 can set a plurality of characteristic points on each reference line. Then, the processing function 112 can calculate the radius of curvature of the approximate circle passing through the characteristic points corresponding to each reference line, and can evaluate the degree of flexion of the shape of the characteristic region with a higher degree of accuracy.

Second Embodiment

In the first embodiment, in the medical image processing apparatus 1, the markers are detected as the characteristic points of the device, and the characteristic region of the device is identified based on the detected characteristic points. Then, in the medical image processing apparatus 1, the registration operation is performed to match the shape of the characteristic region in the other sets of X-ray image data to the shape of the characteristic region in the reference image data. However, the embodiments are not limited to that case. In a second embodiment, the explanation is given about the operation performed in the medical image processing apparatus 1 in the case in which the characteristic shape of a wire-shaped device is detected, and such X-ray images are sequentially updated and displayed in which the region of that characteristic shape or the neighborhood thereof is set and position-adjusted. Meanwhile, a wire-shaped device is a catheter or a guide wire used in the intravascular treatment. In the following explanation, a wire-shaped device is referred to as a wire.

The medical image processing apparatus 1 according to the second embodiment has a fundamentally identical configuration to the medical image processing apparatus 1 illustrated in FIG. 1. However, some of the operations performed therein are different. More particularly, as compared to the medical image processing apparatus 1 according to the first embodiment, in the medical image processing apparatus 1 according to the second embodiment, the operations performed by the processing function 112 and the display control function 114 are different. The following explanation is given with the focus on the differences with the first embodiment.

Regarding the characteristic region of the wire captured in a plurality of sets of image data which is collected in the period of time having less pulsation-induced movement and which has the wire visualized therein, the processing function 112 modifies the shape of the wire captured in the other sets of X-ray image data other than the reference image data in accordance with the shape of the characteristic region of the wire captured in the reference image data. With that, the processing function 112 performs the shape modification operation (the registration operation P2) for matching the shape of a plurality of characteristic regions of the wire.

The characteristic shape of the wire is, for example, the portion visualized to be particularly dark in the guide wire visualized in a set of X-ray image data. More particularly, there are times when, at the leading end portion of the guide wire, a structure having a greater diameter than the other portion of the guide wire is used or a high-density material is used. That enables the operator to grasp the position of the leading end of the guide wire. At that time, as compared to the X-rays that have passed through the other portion of the guide wire, the X-rays that have passed through the leading end portion of the guide wire undergo significant attenuation and are visualized to be dark in the X-ray image data.

Meanwhile, the characteristic shape of the wire also includes a portion having a characteristic shape which changes along the shape of a blood vessel when the wire is inserted in the blood vessel. More particularly, in a set of X-ray image data, there are times when a characteristic contour shape is formed. In the second embodiment, the characteristic contour shape is assumed to be one of the characteristic shapes of the wire.

In that regard, the processing function 112 detects the characteristic shape of the wire, and treats the region in which that shape is visualized or the neighborhood of that region as the characteristic region of the device. Then, in an identical manner to the first embodiment, the processing function 112 performs the registration in such a way that the characteristic shape of the wire in the other sets of X-ray image data are matched to the characteristic region of the wire in the reference image data. Meanwhile, the method for detecting the characteristic shape of the wire can be a known method such as extracting the portion of the wire having a large curvature. Meanwhile, when a plurality of guide wires is inserted and is visualized in a set of X-ray image data, the processing function 112 can detect the characteristic shapes of a plurality of wires and adjust the position of each wire.

The display control function 114 sequentially updates the sets of corrected-image data that are sequentially generated based on the registration operation, and displays the updated sets of corrected-image data in the display 13. Thus, the display control function 114 sequentially displays the sets of X-ray image data, which have less movement of the wire, in the position-adjusted state. Hence, the movement of the wire in the sets of X-ray image data can be held down at the time of display. As a result, in the medical image processing apparatus 1, the wire-shaped device becomes easier to observe, thereby enabling achieving enhancement in the visibility of the wire-shaped device.

Other Embodiments

In the first and second embodiment, the explanation is given about an example in which the image processing is performed with respect to the sets of X-ray image data that are sequentially collected during a partial period of time in a cardiac phase of the subject who has a device inserted into the body. However, the embodiments are not limited by that example. Alternatively, in the medical image processing apparatus 1, the image processing can be performed with respect the sets of X-ray image data that are sequentially generated regarding the subject who has a device inserted into the body. More particularly, with respect to the sets of X-ray image data that are sequentially collected regardless of the cardiac phase, the processing function 112 according to another embodiment can identify the characteristic region of the device captured in the sets of X-ray image data, and can perform the shape modification operation and the addition operation for matching the shape of the other characteristic regions to the shape of the characteristic region captured in the reference image.

As a result, in the medical image processing apparatus 1, irrespective of whether or not a plurality of sets of obtained X-ray image data is collected in a particular cardiac phase, the registration is performed with respect to the shapes of the characteristic regions in the sets of X-ray image data. Hence, it becomes possible to provide X-ray images having further reduction in the changes in the device captured therein. That enables achieving enhancement in the visibility of the device in the sets of X-ray image data.

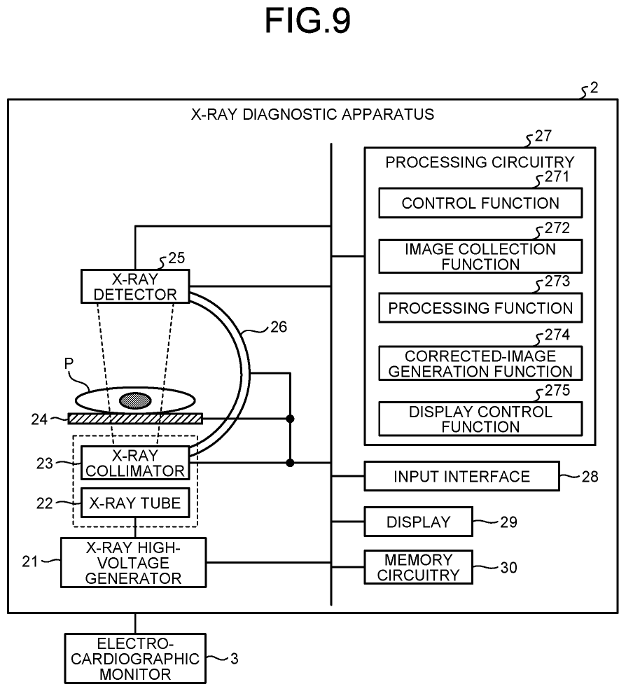

Moreover, in the first and second embodiments, the explanation is given about an example in which the medical image processing apparatus 1 according to the application concerned performs the image processing. However, the embodiments are not limited to that example. Alternatively, the X-ray diagnostic apparatus 2 can perform the image processing. FIG. 9 is a block diagram illustrating an exemplary configuration of the X-ray diagnostic apparatus 2 according to the other embodiment. As illustrated in FIG. 9, the X-ray diagnostic apparatus 2 is connected to an electrocardiographic monitor 3.

The X-ray diagnostic apparatus 2 collects the X-ray image data from a subject P. For example, during the period of time in which a device is inserted into the subject P and a procedure is being performed, if an X-ray irradiation instruction is received from the operator via an input interface 28, then the X-ray diagnostic apparatus 2 performs X-ray irradiation and collects sets of X-ray image data. Then, the X-ray diagnostic apparatus 2 performs the image processing such as the registration operation and the addition operation performed according to the application concerned with respect to the sets of X-ray image data, and displays the processed image data in a display 29.

The X-ray diagnostic apparatus 2 includes an X-ray high-voltage generator 21, an X-ray tube 22, an X-ray collimator 23, a tabletop 24, an X-ray detector 25, a C-arm 26, the processing circuitry 27, the input interface 28, a display 29, and memory circuitry 30. The X-ray high-voltage generator 21 generates a high voltage under the control of the processing circuitry 27 and applies the high voltage to the X-ray tube 22. Based on the high voltage applied by the X-ray high-voltage generator 21, the X-ray tube 22 irradiates the subject P with X-rays who is present on the tabletop 24. Under the control of the processing circuitry 27, the X-ray collimator 23 opens and closes the collimator blade, and forms an exposure range (exposure field) of the X-rays irradiated from the X-ray tube 22. For example, the collimator blade is formed in the shape of a plate using an X-ray shielding material such as lead. The tabletop 24 is a bed on which the subject P is asked to lie down, and is placed on top of a table (not illustrated).

The X-ray detector 25 is an X-ray flat plane detector (FPD) in which, for example, radiation detecting elements are arranged in a matrix. The X-ray detector 25 detects X-rays that, after being irradiated from the X-ray tube 22, have passed through the subject P; and outputs detection signals according to the detected X-ray dosage (i.e., outputs X-ray detection signals) to the processing circuitry 27. The C-arm 26 supports the X-ray tube 22 and the X-ray collimator 23 on one side and supports the X-ray detector 25 on the opposite side across the subject P. The C-arm 26 has a driving mechanism such as a motor and an actuator and, under the control of the processing circuitry 27 (explained later), operates the driving mechanism to perform rotation/movement. With reference to FIG. 9, the explanation is given about an example in which the X-ray diagnostic apparatus 2 is of the single-plane type. However, the embodiments are not limited to that case, and alternatively the X-ray diagnostic apparatus 2 can be of the biplane type.

The input interface 28 according to the other embodiment includes an exposure switch such as a foot switch in addition to including the input interface 12 according to the first and second embodiments. The memory circuitry 30 is used to store a computer program that is configured in an identical manner to the computer program in the medical image processing apparatus 1 according to the first and second embodiments, that is executed by the processing circuitry 27, and that causes the processing circuitry 27 to function as various functions. The display 29 has an identical configuration to the display in the medical image processing apparatus 1 according to the first and second embodiments. Hence, that explanation is not given again.

The processing circuitry 27 reads the computer program stored in the memory circuitry 30, and accordingly functions as a control function 271, an image collection function 272, a processing function 273, a corrected-image generation function 274, and a display control function 275. The processing circuitry 27 is configured using, for example, a processor. The control function 271 supplies control signals to the X-ray high-voltage generator 21, the X-ray collimator 23, the tabletop 24, the X-ray detector 25, and the C-arm 26, so that X-ray irradiation is carried out. The image collection function 272 supplies control signals to the X-ray detector 25, and controls X-ray irradiation. Then, based on the detection signals detected by the X-ray detector 25, the image collection function 272 stores X-ray image data and stores it in the memory circuitry 30. The processing function 273, the corrected-image generation function 274, and the display control function 275 perform identical operations to the processing function 112, the corrected-image generation function 113, and the display control function 114, respectively, explained in the first and second embodiments.

Meanwhile, the term “processor” used in the description of the embodiments implies, for example, a central processing unit (CPU), or a graphics processing unit (GPU), or an application specific integrated circuitry (ASIC), or a programmable logic device (such as a simple programmable logic device (SPLD), or a complex programmable logic device (CPLD), or a field programmable gate array (FPGA)). Moreover, instead of storing a computer program in the memory circuitry 14, it can be directly incorporated into the circuitry of a processor. In that case, the processor reads the computer program incorporated in the circuitry and executes it so that the functions get implemented. Meanwhile, the processors according to the embodiments are not limited to be configured using a single circuitry on a processor-by-processor basis. Alternatively, a single processor can be configured by combining a plurality of independent circuitries, and the corresponding functions can be implemented.

A computer program executed by a processor is stored in advance in a read only memory (ROM) or a memory circuit. Alternatively, the computer program can be recorded as an installable file or an executable file in a non-transitory computer-readable storage medium such as a compact disk read only memory (CD-ROM), a flexible disk (FD), a compact disk recordable (CD-R), or a digital versatile disk (DVD). Still alternatively, the computer program can be stored in a downloadable manner in a computer that is connected to a network such as the Internet. For example, the computer program is configured using modules of the processing functions explained above. As far as the actual hardware is concerned, a CPU reads the computer program from a storage medium such as a ROM and executes it, so that the modules get loaded and generated in a main memory device.

In the embodiments and the modification examples described above, the constituent elements of the device illustrated in the drawings are merely conceptual, and need not be physically configured as illustrated. The constituent elements, as a whole or in part, can be separated or integrated either functionally or physically based on various types of loads or use conditions. The processing functions implemented by the device are entirely or partially implemented by the CPU or by computer programs that are analyzed and executed by the CPU, or are implemented as hardware by wired logic.

Of the processes described in the embodiments, all or part of the processes explained as being performed automatically can be performed manually. Similarly, all or part of the processes explained as being performed manually can be performed automatically by a known method. The processing procedures, the control procedures, specific names, various data, and information including parameters described in the embodiments or illustrated in the drawings can be changed as required unless otherwise specified.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising: processing circuitry configured to
obtain a plurality of X-ray images that are sequentially collected during a partial period of time in a cardiac phase of a subject who has a device inserted into body thereof,
identify characteristic region of the device captured in the plurality of X-ray images that is obtained, and
perform registration in which
position of the characteristic region identified in a reference image, which is one of the plurality of X-ray images, serves as reference position, and
position of the characteristic region identified in the plurality of X-ray images collected after the reference image is adjusted based on the reference position.

2. The medical image processing apparatus according to claim 1, wherein the processing circuitry is configured to
generate, based on the registration, a corrected image from an X-ray image collected after the reference image, and
display, in a display, an added image generated by adding a plurality of the corrected image.

3. The medical image processing apparatus according to claim 1, wherein the processing circuitry is configured to
sequentially generate, based on the registration, a correct image from an X-ray image collected after the reference image, and
sequentially update the corrected image and display the updated corrected image in a display.

4. The medical image processing apparatus according to claim 1, wherein the processing circuitry is configured to obtain, based on electrocardiogram information, a plurality of X-ray images collected as a result of irradiation of X-rays during the partial period of time of the cardiac pulsation from a plurality of X-ray images sequentially generated after insertion of the device,.

5. The medical image processing apparatus according to claim 1, wherein the processing circuitry is configured to perform the registration by performing a shape modification operation in which, regarding a plurality of identified characteristic regions of the device, shape of characteristic region of the device captured in an X-ray image other than a reference image from among the plurality of X-ray images is modified in accordance with shape of characteristic region of the device captured in the reference image from among the plurality of X-ray images.

6. The medical image processing apparatus according to claim 5, wherein the processing circuitry is configured to perform the shape modification operation by
setting, with respect to a characteristic region captured in the plurality of X-ray images, a plurality of characteristic points reflecting shape of the characteristic region, and
matching or approximating a plurality of characteristic points set in characteristic region captured in an X-ray image other than the reference image to a plurality of characteristic points set in characteristic region in the reference image.

7. The medical image processing apparatus according to claim 6, wherein the processing circuitry is configured to
detect at least one of characteristic objects of the device,
identify, as characteristic region of the device, a region in the X-ray image in which the characteristic object is captured, and
set positions of the plurality of characteristic points in the X-ray image according to the characteristic object captured in the characteristic region and according to shape of the characteristic region.

8. The medical image processing apparatus according to claim 5, wherein the processing circuitry is configured to
evaluate degree of flexion of shape of each of a plurality of characteristic regions of the device, and
depending on evaluation result, exclude shape of the characteristic region from target for the shape modification operation.

9. The medical image processing apparatus according to claim 1, wherein, among the plurality of X-ray images obtained, the processing circuitry is configured to set, as the reference image, an image that is initially collected during the partial period of time in the cardiac phase.

10. The medical image processing apparatus according to claim 1, wherein the processing circuitry is configured to set, as the reference image, an image decided according to an operation performed by an operator.

11. The medical image processing apparatus according to claim 1, wherein, based on shapes of a plurality of characteristic regions of the device, the processing circuitry is configured to evaluate degree of flexion of each of the shapes and set, as the reference image, an image having least flexion of the device based on evaluation result.

12. The medical image processing apparatus according to claim 1, wherein the partial period of time in the cardiac phase is a period of time in which there is relatively less movement in cardiac pulsation of the subject.

13. A medical image processing apparatus comprising processing circuitry configured to
obtain an X-ray image that is sequentially generated regarding a subject who has a device inserted into body thereof,
identify characteristic region of the device captured in each of a plurality of X-ray images that is obtained,
perform a shape modification operation for
modifying, in a plurality of identified characteristic regions of the device, shape of characteristic region of the device captured in an X-ray image, from among the plurality of X-ray images, other than a reference image, which is one of the plurality of X-ray images, to shape of characteristic region of the device captured in the reference image, and
matching shape of the plurality of characteristic regions of the device, and
add, to the reference image, a plurality of shape-modified images generated from X-ray images other than the reference image based on the shape modification operation, and display generated added image in a display.

14. An X-ray diagnostic apparatus comprising:
an X-ray tube configured to irradiate a subject, who has a device inserted into body thereof, with X-rays during a partial period of time in a cardiac phase of the subject;
an X-ray detector configured to detect X-rays which have passed through the subject; and
processing circuitry configured to
control the X-ray tube and the X-ray detector, and collect an X-ray image based on the X-rays detected by the X-ray detector,
identify characteristic region of the device captured in a plurality of X-ray images collected during the partial period of time in cardiac phase of the subject, and
perform registration in which
position of the characteristic region identified in a reference image, which is one of the plurality of X-ray images, serves as reference position, and
position of the characteristic region identified in the plurality of X-ray images collected after the reference image is adjusted based on the reference position.

15. The X-ray diagnostic apparatus according to claim 14, wherein the processing circuitry is configured to
identify the partial period of time in the cardiac phase based on electrocardiogram information of the subject, and
control the X-ray tube and the X-ray detector to ensure that X-ray irradiation occurs during the identified partial period of time and that the plurality of X-ray images are collected.

16. An X-ray diagnostic apparatus comprising:
an X-ray tube configured to irradiate a subject, who has a device inserted into body thereof, with X-rays;
an X-ray detector configured to detect X-rays which have passed through the subject; and
processing circuitry configured to
collect an X-ray image, which is sequentially generated, based on the detected X-rays,
identify characteristic region of the device captured in each of a plurality of X-ray images that is collected,
perform a shape modification operation for
modifying, in a plurality of identified characteristic regions of the device, shape of characteristic region of the device captured in an X-ray image, from among the plurality of images, other than a reference image, which is one of the plurality of X-ray images, to shape of characteristic region of the device captured in the reference image, and
matching shape of the plurality of characteristic regions of the device, and
add, to the reference image, a plurality of shape-modified images generated from X-ray images other than the reference image based on the shape modification operation, and display generated added image in a display.

17. A storage medium storing therein, in a non-transitory manner, a program that causes a computer to execute processes of:
identifying characteristic region in a device which is inserted into body of a subject and which is captured in a plurality of X-ray images sequentially collected during a partial period of time in a cardiac phase of the subject; and
performing registration in which
position of the characteristic region identified in a reference image, which is one of the plurality of X-ray images, serves as reference position, and
position of the characteristic region identified in the plurality of X-ray images collected after the reference image is adjusted based on the reference position.

18. A storage medium storing therein, in a non-transitory manner, a program that causes a computer to execute processes of:
identifying a characteristic region of a device captured in each of a plurality of X-ray images that is sequentially created regarding a subject who has the device inserted into body;
performing a shape modification operation for
modifying, in a plurality of identified characteristic regions of the device, shape of characteristic region of the device captured in an X-ray image, from among the plurality of X-ray images, other than a reference image, which is one of the plurality of X-ray images, to shape of characteristic region of the device captured in the reference image, and
matching shape of the plurality of characteristic regions of the device; and
adding that includes adding, to the reference image, a plurality of shape-modified images generated from X-ray images other than the reference image based on the shape modification operation, and displaying generated added image in a display unit.
